(19) United States
(12) Patent Application Publication
(10) Pub. No.: US 2022/0361859 A1
(43) Pub. Date: Nov. 17, 2022

(54) Devices, Systems, and Methods for Positioning an Elongate Member within a Body Lumen

(71) Applicant: BOSTON SCIENTIFIC SCIMED INC., Maple Grove, MN (US)

(72) Inventors: Diana Catalina RODRIGUEZ FORERO, Eircode (IE); Gonzalo Jose SAENZ VILLALOBOS, Alajuela (CR); Kevin MCEVILLY, Galway (IE); Elias Milad SIBANI, El Coyol (CR); Daniel CALVO CAMACHO, Ciudad Colonc (CR); Alejandro BRENES ACOSTA, Watertown, MA (US)

(73) Assignee: BOSTON SCIENTIFIC SCIMED INC, Maple Grove, MN (US)

(21) Appl. No.: 17/742,699

(22) Filed: May 12, 2022

Related U.S. Application Data
(60) Provisional application No. 63/187,664, filed on May 12, 2021.

(51) Int. Cl. A61B 10/04; A61B 10/02; A61B 1/00; A61B 17/34; A61B 1/267

(52) U.S. Cl. CPC A61B 10/04; A61B 10/0233; A61B 1/00098; A61B 17/3403; A61B 1/267; A61B 2017/3413

(57) ABSTRACT

The present disclosure relates generally to positioning elongate members at a target site within a body lumen, such as for acquiring a biopsy from a peripheral airway. Some embodiments are particularly directed to an elongate member with an embedded transducer positioned at a predefined rotational angle with respect to a projected position of an instrument extended out of a distal opening of a first lumen in the elongate member. In many such embodiments, a rotational transducer may be positioned within a second lumen in the elongate member to generate a radial image including indicia of the embedded transducer. Accordingly, an operator may determine a projected position of the instrument prior to extending the instrument out of the lumen. In several embodiments, the embedded transducer may include a forward imaging transducer, such as a fiber optic.

### FIELD

The present disclosure relates generally to the field of medical devices. In particular, the present disclosure relates to devices, systems, and methods to facilitate positioning an elongate member at a target site within a body lumen.

### BACKGROUND

A variety of medical devices are positioned within a body lumen for diagnostic or therapeutic purposes. For example, an endoscopy is a procedure using an endoscope to look inside a body. Typically, an endoscopy procedure utilizes an elongate member (e.g., an endoscope) to access, examine, or interact with the interior of a hollow organ or cavity of a body for diagnostic or therapeutic purposes. The endoscope typically has direct visualization for viewing inside the body and/or may be equipped with ultrasound view capability. Such scopes have a profile diameter that allow the scope to be inserted into larger body lumens (e.g., gastrointestinal (GI) tract or trachea) of a certain diameter. For example, one type of endoscope, a bronchoscope can be used for visualizing the inside of the airways, up to a certain generation of airway having a diameter that can accommodate the diameter of the bronchoscope, for diagnostic and therapeutic purposes. The bronchoscope is inserted into the airways through a mouth, nose, or tracheostomy. This may allow the practitioner to examine the patient's airways for abnormalities such as foreign bodies, bleeding, tumors, or inflammation. Sometimes a biopsy may be taken from inside the lungs. At a certain higher generations of airways, the diameter of the airway becomes too narrow to accommodate conventional bronchoscopes, which presents the challenge for improved devices having means to accurately navigate, locate, and biopsy tissue within these smaller airways or within other lumens of minimal diameter.

### SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In one aspect, the present disclosure relates to a medical device with an elongate member and a rotational transducer. The elongate member may have a proximal end, a distal end, a first lumen extending from the proximal end to a first distal opening proximate the distal end, a second lumen extending from the proximal end to the distal end, and an embedded transducer disposed in a wall of the elongate member. The embedded transducer may be positioned with a predefined rotational angle with respect to a projected position of an instrument extended out of a distal opening of the first lumen. The rotational transducer may be disposed in the second lumen and configured to generate a radial image from within the second lumen. The radial image may include indicia of the embedded transducer at the predefined rotational angle with respect to the projected position of the instrument.

In some embodiments, the embedded transducer includes at least a portion of an imaging transducer. In some such embodiments, the embedded transducer comprises a distal portion of a fiber optic sensor. In various embodiments, the rotational transducer comprises a rotational imaging transducer. In various such embodiments, the rotational imaging transducer may include an ultrasound transducer. In several embodiments, the embedded transducer is disposed within a wall of the second lumen of the elongate member. In many embodiments, the second lumen is positioned in the elongate member between the first lumen and the embedded transducer. In some embodiments, the instrument comprises a pre-curved instrument. In some such embodiments, the instrument comprises one or both of a needle and an ablation probe. In various such embodiments, the elongate member is configured to bend in a first direction when a distal end of the pre-curved instrument is positioned a first distance from the distal opening of the first lumen. In many further such embodiments, the elongate member is configured to straighten as the pre-curved instrument is moved from the first distance to the distal opening of the first lumen. In several further such embodiments, the elongate member is configured to remain straight (or substantially straight) when the pre-curved instrument is extended out of the distal opening of the first lumen. In various embodiments, the predefined rotational angle ranges from about 45 to about 315 degrees. In many embodiments, the elongate member has an outer diameter of less than 2 mm.

In another aspect, the present disclosure relates to a system comprising an elongate member, an instrument, and a rotational transducer. The elongate member may have a proximal end, a distal end, a first lumen, a second lumen, and an embedded transducer. The instrument may be disposed in the second lumen. The embedded transducer may be positioned with a predefined rotational angle with respect to a projected position of the instrument extended out of a distal opening of the first lumen. The rotational transducer may be disposed in the second lumen, The rotational transducer may be configured to generate a radial image from within the second lumen. The radial image may include indicia of the embedded transducer at the predefined rotational angle with respect to the projected position of the instrument.

In some embodiments, the instrument comprises a pre-curved needle. In various embodiments, the instrument comprises a pre-curved ablation probe.

In yet another aspect, the present disclosure relates to a method. The method may include inserting a distal end of an elongate member into a body lumen. The elongate member may have a proximal end, the distal end, a first lumen, a second lumen, and an embedded transducer. The method may include generating a radial image with a rotational transducer disposed in the second lumen of the elongate member. The radial image may include indicia of the embedded transducer at a predefined rotational angle with respect to a projected position of an instrument extended out of a distal opening of the first lumen of the elongate member.

In several embodiments, the method includes rotating the elongate member to align a target tissue at the predefined rotational angle with respect the projected position of the instrument. In several embodiments, the method includes extending the instrument out of the distal opening of the first lumen of the elongate member to obtain a biopsy of the target tissue.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. In will be appreciated that various figures included in this disclosure may omit some components, illustrate portions of some components, and/or present some components as transparent to facilitate illustration and description of components that may otherwise appear hidden. For purposes of clarity, not every component is labeled in every figure, nor is every component of each embodiment shown where illustration is not necessary to allow those of ordinary skill in the art to understand the disclosure. In the figures:

FIG. 1 illustrates an exemplary medical device according to one or more embodiments disclosed hereby.

FIGS. 2A-2C illustrate various aspects of steering according to one or more embodiments disclosed hereby.

FIGS. 3A-3C illustrate various aspects of imaging according to one or more embodiments disclosed hereby.

### DETAILED DESCRIPTION

The present disclosure relates generally to positioning elongate members at a target site within a body lumen, such as for acquiring a biopsy from a peripheral airway. Some embodiments are particularly directed to an elongate member with an embedded transducer positioned at a predefined rotational angle with respect to a projected position of an instrument extended out of a distal opening of a first lumen in the elongate member. In many such embodiments, a rotational transducer may be positioned within a second lumen in the elongate member to generate a radial image including indicia of the embedded transducer. Accordingly, an operator may determine a projected position of the instrument prior to extending the instrument out of the lumen. In several embodiments, the embedded transducer may include a forward imaging transducer, such as a fiber optic. In various embodiments, the instrument may have a predefined curve. In various such embodiments, the predefined curve of the instrument may be utilized to steer the elongate member. For example, a deflection angle of the elongate member may be adjusted by varying the distance of the pre-curved instrument with respect to the distal end of the elongate member. Additionally, one or more of these features may be combined into an elongate member with small enough dimensions to access narrow peripheral body lumens. These and other embodiments are described and claimed.

There are challenges with positioning elongate members of medical devices at target sites within a body lumen, such as external dimensions that limit access to narrow body lumens. For example, endobronchial ultrasound (EBUS) scopes are too large (e.g., scope flex tube OD over 4.2 mm) to reach into certain peripheral portions of body lumens (e.g., certain generations of peripheral airways), where suspected cancerous nodules may be located. Electromagnetic (EM) bronchoscopy may be used to locate targets sites (e.g., suspected cancerous nodules in the periphery of the airway). However, errors, such as those introduced through a combination of metallic distortion and pre-operative computerized tomography (CT) scans to intraoperative patient position divergence, can complicate confirmation of proper positioning at a target site. Accordingly, operators may rely on other technologies, such as reusable, single element (rotational) ultrasound probes, used in conjunction with EM bronchoscopy to confirm proper positioning at a target site. However, these probes are typically non-steerable and fill an entire lumen (e.g., working channel), necessitating removal of the probe before an instrument (e.g., a biopsy needle) can be inserted into the lumen. Further, device exchanges can contribute to tip movement and removal of the ultrasound probe makes confirming the location of the elongate member once the instrument has been inserted uncertain, leading to a number of challenges, such as lower diagnostic yield for biopsies. Such limitations can reduce the usability and applicability of medical devices for positioning elongate member at target sites, contributing in some cases to inefficient devices with limited capabilities. It is with these considerations in mind that a variety of advantageous medical outcomes may be realized by the devices, systems, and methods of the present disclosure.

The following detailed description should be read with reference to the drawings, which depict illustrative embodiments. The present disclosure is not limited to the particular embodiments described, as such embodiments may vary. The terminology used herein is for the purpose of describing particular embodiments only and is not intended to be limiting beyond the scope of the appended claims. Unless defined otherwise, all technical terms used herein have the same meaning as commonly understood by one of ordinary skill in the art to which the disclosure belongs. Finally, although embodiments of the present disclosure may be described with specific reference to medical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like. The structures and configurations, and methods of deploying, in order to stabilize, maintain, and/or otherwise facilitate fluid flow paths may find utility beyond treatments discussed herein.

As used herein, “proximal end” refers to the end of a device that lies closest to the user (medical professional or clinician or technician or operator or physician, etc., such terms being used interchangeably herein without intent to limit, and including automated controller systems or otherwise) along the device when introducing the device into a patient, and “distal end” refers to the end of a device or object that lies furthest from the user along the device during implantation, positioning, or delivery.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise.

All numeric values are herein assumed to be modified by the term “about,” whether or not explicitly indicated. The term “about,” in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified. The recitation of numerical ranges or values by endpoints includes all numbers within that range, including the endpoints (e.g. 1 to 5 includes 1, 1.5, 2, 2.75, 3, 3.80, 4, and 5), and fractions thereof.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

It may be understood that the disclosure included herein is exemplary and explanatory only and is not restrictive. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Although endoscopes and endoscopic systems are referenced herein, reference to endoscopes, endoscopic systems, or endoscopy should not be construed as limiting the possible applications of the disclosed aspects. For example, the disclosed aspects may be used in conjunction with duodenoscopes, bronchoscopes, ureteroscopes, colonoscopes, catheters, diagnostic or therapeutic tools or devices, or other types of medical devices or systems.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

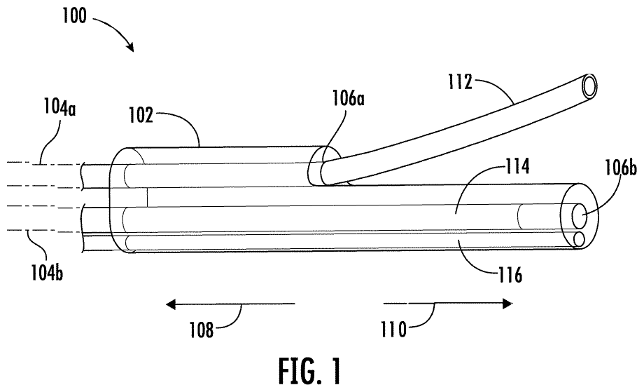

FIG. 1 illustrates a medical device 100 according to one or more embodiments disclosed hereby. Medical device 100 includes an elongate member 102, an instrument 112, and a rotational transducer 114. The elongate member 102 may include a proximal end 108, a distal end 110, a first lumen 104a extending from the proximal end 108 end to distal opening 106a, a second lumen 104b extending from the proximal end 108 to the distal opening 106b, and an embedded transducer 116. In some embodiments, the embedded transducer 116 may comprise a forward-imaging transducer embedded in a wall of elongate member 102. In many embodiments, the rotational transducer 114 may include a radial imaging transducer. In some embodiments, FIG. 1 may include one or more components that are the same or similar to one or more other components of the present disclosure. Further, one or more components of FIG. 1, or aspects thereof, may be incorporated into other embodiments of the present disclosure, or excluded from the described embodiments, without departing from the scope of this disclosure. For example, embodiments of medical device 100 may exclude instrument 112 without departing from the scope of this disclosure. Still further, one or more components of other embodiments of the present disclosure, or aspects thereof, may be incorporated into one or more components of FIG. 1, without departing from the scope of this disclosure. Embodiments are not limited in this context.

In various embodiments, the proximal end 108 of elongate member 102 may include, or be coupled to, one or more controllers and/or user interfaces. For example, medical device 100 may include a controller communicatively coupled to the embedded transducer 116 and/or rotational transducer 114. The controller may provide an interface that allows an operator to control and monitor the rotational transducer 114 and/or embedded transducer 116. In some embodiments, the controller may provide torque to rotate the rotational transducer 114.

In many embodiments, embedded transducer 116 is positioned at a predefined rotational angle with respect to a projected position of instrument 112 extended out of distal opening 106a to lumen 104a in the elongate member 102. For example, embedded transducer 116 may extend through the field of view of rotational transducer 114 at the predefined rotational angle with respect to the projected position of instrument 112 extended out of a distal opening 106a to lumen 104a. Further, embedded transducer 116, or at least the portion extending through the field of view of rotational transducer 114 may be constructed from a material that will cause indicia in the radial image (e.g., a material that interacts with imaging energy emitted by rotational transducer 114 in a characteristic way).

In various embodiments, the lumens 104a, 104b may extend from the proximal end 108 of elongate member 102 to distal openings 106a, 106b, respectively. In several embodiments, the lumen 104a may terminate prior to the lumen 104b. In several such embodiments, terminating lumen 104a prior to lumen 104b may facilitate imaging a projected position of instrument 112 with rotational transducer 114 while rotational transducer 114 remains within lumen 104b. Accordingly, embedded transducer 116 can cause indicia in the radial image indicating the projected position of instrument 112. In some embodiments, distal openings 104a, 104b may be parallel with respect to each other. In another embodiment, distal openings 104a, 104b may be perpendicular with respect to each other.

In several embodiments, the embedded transducer 116 may be disposed in a wall of the elongate member 102. For example, the embedded transducer 116 may be disposed in a wall of the second lumen 104b. In another example, the embedded transducer 116 may be disposed in a wall of the first lumen 104a. It will be appreciated that the embedded transducer 116 may be disposed in a wall of the elongate member 102 to position the embedded transducer 116 at the predefined rotational angle with respect to the projected of instrument 112.

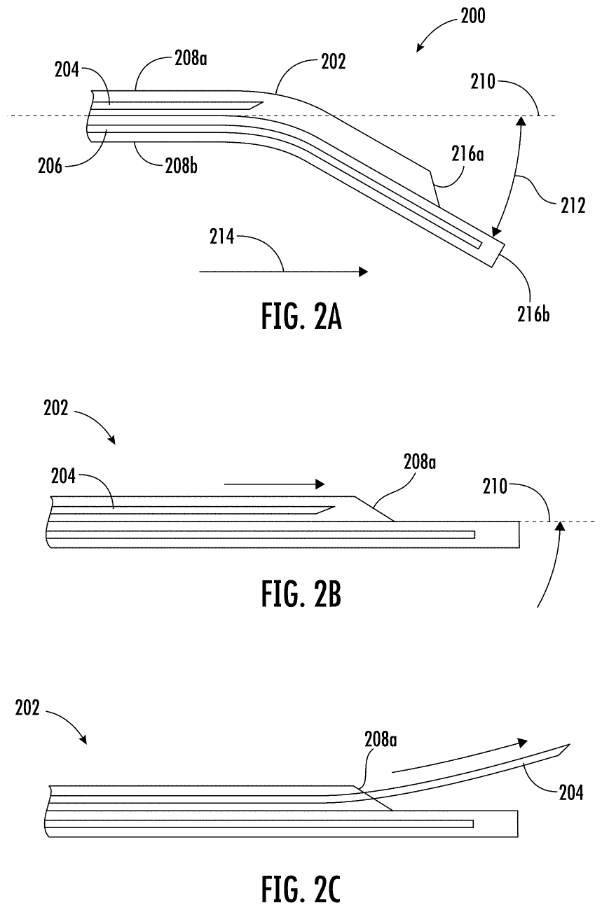

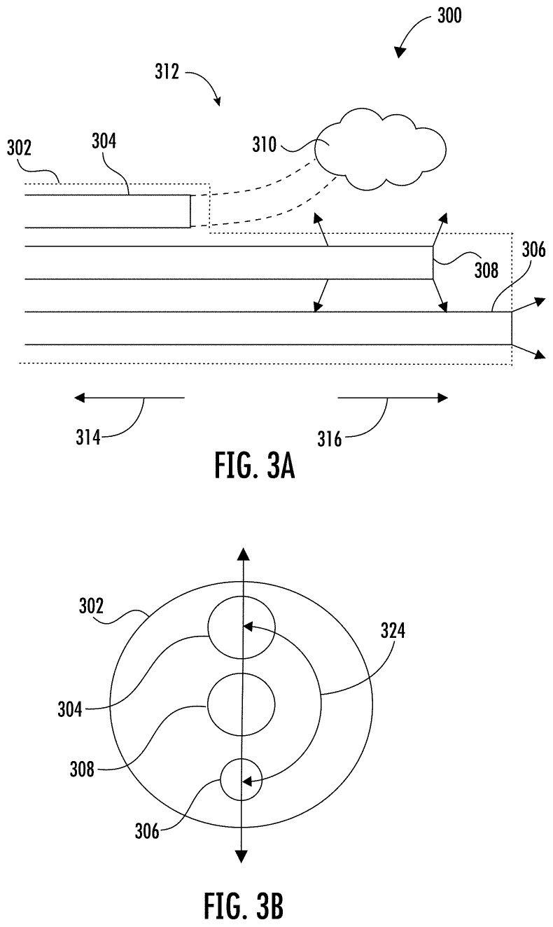

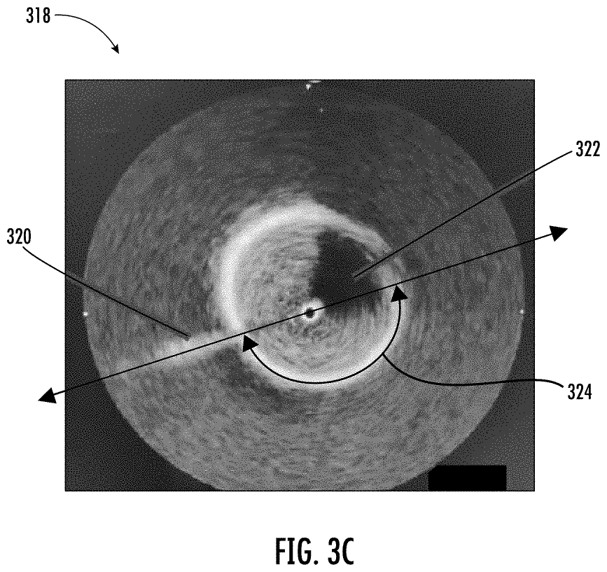

As will be described in more detail below, such as with respect to FIGS. 3A-3C, in some embodiments, the predefined rotational angle may range from about 45 to about 315 degrees. For example, the predefined rotational angle may be about 180 degrees. In several embodiments, rotational transducer 114 may be positioned within lumen 104b to generate a radial image including indicia of the embedded transducer 116. Accordingly, an operator may determine a projected position of the instrument 112 prior to extending the instrument 112 out of the lumen 104a. As will be described in more detail below, such as with respect to FIGS. 2A-2C, in many embodiments, instrument 112 may additionally, or alternatively, be utilized to steer the distal end 110 of elongate member 102.

In one or more embodiments, the embedded transducer 116 may comprise at least a portion (e.g., a distal portion) of an imaging transducer, such as a forward-imaging transducer. For example, embedded transducer 116 may refer to or include a waveguide, such as a fiber optic cable, that is coupled to, or included in, a fiber optic sensor. In some such examples, at least a portion of the fiber optic sensor may not be embedded in the elongate member, such as by extending out of a distal end of the elongate member 102. In one embodiment, the embedded transducer 116 may comprise a distal portion of a fiber optic sensor. In various embodiments, the embedded transducer 116 may include a plurality of wave guides. For example, a first wave guide may be utilized for collecting light from the distal end 110 (e.g., for imaging) and a second wave guide may be utilized for providing light to the distal end 110 (e.g., for lighting). In other examples, a single wave guide may be alternated between collecting and providing light. In various embodiments, lumen 104b may extend further distally than lumen 104a. In various such embodiments, this may facilitate imaging a projected position of instrument 112 while rotational transducer 114 is disposed within lumen 104a.

In many embodiments, embedding the transducer 116 may enable an orientation of the embedded transducer 116 to remain fixed and known. Accordingly, the orientation of the embedded transducer 116 relative to the rotational transducer 114 (and in radial images generated thereby) can be readily determined and utilized to aid in navigation. For example, when embedded transducer 116 comprises a forward-imaging transducer, radial images generated by rotational transducer 114 that include indicia of the embedded transducer 116 may be used to a dome shaped image surrounding the distal end of elongate member 100.

In various embodiments, the outer diameter of the elongate member 102 may be less than 2 mm, such as 1.5 mm or 1.9 mm. In one or more embodiments, the outer diameter of the rotational transducer 114 may be less than 1.3 mm, such as 1.1 mm. In some embodiments, the rotational transducer 114 may comprise a radial endobronchial ultrasound (rEBUS) probe. In some such embodiments, the rEBUS probe may operate at between 10 and 70 hertz, such as 40 hertz. In many embodiments, the instrument 112 may include a biopsy needle, such as for transbronchial-needle aspiration (TBNA). In many such embodiments, the instrument 112 may comprise a needle between 10 and 40 gauge, such as a 25 gauge needle.

In several embodiments, transducer may generally refer to a device that converts energy from one form into another. In many embodiments, each transducer may operate to convert one or more electrical signal to one or more physical quantities (e.g., energy, force, torque, light, motion, position, etcetera) and/or convert one or more physical quantities to one or more electrical signals. For example, a transducer (e.g., rotational transducer 114 and/or embedded transducer 116) may include one or more of an imaging sensor, a phased array sensor, a position sensor, a light emitting diode, a pressure sensor, an actuator, an inductive sensor, a fiber-optic sensor, an electromagnetic position sensor, or the like.

FIGS. 2A-2C illustrate various aspects of steering a medical device 200 according to one or more embodiments disclosed hereby. The medical device 200 includes an elongate member 202, a pre-curved instrument 204, and rotational transducer 206. Elongate member 202 has a distal end 214 and includes lumen 208a having a distal opening 216a and lumen 208b having distal opening 216b. In some embodiments, FIGS. 2A-2C may include one or more components that are the same or similar to one or more other components of the present disclosure. For example, elongate member 202 may be the same or similar to elongate member 102. Further, one or more components of FIGS. 2A-2C, or aspects thereof, may be incorporated into other embodiments of the present disclosure, or excluded from the described embodiments, without departing from the scope of this disclosure. For example, pre-curved instrument 204 may be incorporated into medical device 100 without departing from the scope of this disclosure. Still further, one or more components of other embodiments of the present disclosure, or aspects thereof, may be incorporated into one or more components of FIG. 2A-2C, without departing from the scope of this disclosure. For example, embedded transducer 116 may be incorporated into elongate member 202 without departing from the scope of this disclosure. Embodiments are not limited in this context.

In FIG. 2A, pre-curved instrument 204 is disposed within lumen 208a at a first distance from distal opening 216a, and elongate member 202 is deflected an angle 212 with respect to the horizontal axis 210. In FIG. 2B, pre-curved instrument 204 is disposed within lumen 208a at a second distance from distal opening 216b, and elongate member 202 is aligned with horizontal axis 210. In FIG. 2C, pre-curved instrument 204 is extended out of distal opening 216b, and elongate member 202 is aligned with horizontal axis 210. In other words, the elongate member 202 may be configured to remain straight (or substantially straight) when the pre-curved instrument is extended out of the distal opening of the first lumen. In various embodiments described herein, the deflection of elongate member 202 may be used for steering. For example, angle 212 combined with rotation of the elongate member 202 may be utilized to position the distal end 214 of elongate member 202 with respect to a target tissue. In such examples, imaging with one or more transducers included in elongate member 202 provide guidance utilized in positioning the distal end 214 of elongate member 202 with respect to the target tissue. In various embodiments, the angle 212 may be adjusted by varying the distance of the pre-curved instrument 204 with respect to the distal end of the elongate member 202.

In several embodiments, the angle 212 may be adjusted continuously between a minimum and maximum angles by varying the distance of the pre-curved instrument 204 with respect to the distal end 214 of the elongate member 202. In many embodiments, indicia of a distance of the pre-curved instrument 204 with respect to the distal end 214 of the elongate member 202 may be provided in radial images. For example, equally spaced strips along lumen 208a may provide, in radial images, indicia of the distance of the pre-curved instrument 204 with respect to the distal end 214 of the elongate member 202. In one or more embodiments, the elongate member 202 may have a predefined curvature. For example, FIG. 2A may illustrate a predefined curvature of elongate member 202. It will be appreciated that some embodiments may utilize a pre-curved instrument 204, such as for steering, without including an embedded transducer and/or a rotational transducer.

FIGS. 3A-3C illustrate various aspects of imaging with a medical device 300 according to one or more embodiments disclosed hereby. The medical device 300 includes an elongate member 302 with proximal end 314 and distal end 316, pre-curved instrument 304, embedded imaging transducer 306, and rotational imaging transducer 308. Additionally, medical device 300 may utilize rotational imaging transducer 308 to generate radial image 318. In some embodiments, FIGS. 3A-3C may include one or more components that are the same or similar to one or more other components of the present disclosure. For example, elongate member 302 may be the same or similar to elongate member 202. Further, one or more components of FIGS. 3A-3C, or aspects thereof, may be incorporated into other embodiments of the present disclosure, or excluded from the described embodiments, without departing from the scope of this disclosure. For example, embedded imaging transducer 306 may be integrated into elongate member 202 without departing from the scope of this disclosure. Still further, one or more components of other embodiments of the present disclosure, or aspects thereof, may be incorporated into one or more components of FIG. 3A-3C, without departing from the scope of this disclosure. For example, one or more aspects of steerability discussed with respect to FIGS. 2A-2C may be incorporated into medical device 300 without departing from the scope of this disclosure. Embodiments are not limited in this context.

FIG. 3A includes a side view of medical device 300 disposed within a body lumen 312. More specifically, FIG. 3A may illustrate the fields of view of embedded imaging transducer 306 and rotational imaging transducer 308 that can be utilized in positioning the distal end 316 of elongate member 302 with respect to target tissue 310. Additionally, the illustrated embodiment includes a projected position of pre-curved instrument 304 when extended out of elongate member 302. In various embodiments, medical device 300 may utilize embedded imaging transducer 306 and rotational imaging transducer 308 to enable an operator to position elongate member 302 with respect to target tissue 310, such as to obtain a biopsy or delivery an ablation therapy with pre-curved instrument 304. Accordingly, in various embodiments, pre-curved instrument 304 may include a biopsy needle or an ablation probe. Further, in several embodiments, the steerability discussed previously may be combined with the imaging capabilities to provide a medical device capable of navigating to peripheral airways and obtain biopsies or delivery ablation therapy in efficient and economical manners. In several embodiments, medical devices described hereby may be utilized to obtain biopsies of or deliver a therapy to nodules in peripheral airways of lungs. It will be appreciated that some embodiments may utilize an embedded transducer and/or a rotational transducer without including a pre-curved instrument 204, such as for steering.

In various embodiments, medical device 300 may utilize embedded imaging transducer 306 and rotational imaging transducer 308 to enable an operator to position elongate member 302 with respect to target tissue 310, such as to obtain a biopsy with pre-curved instrument 304. Accordingly, in various embodiments, pre-curved instrument 304 may include a biopsy needle. Further, in several embodiments, the steerability discussed previously may be combined with the imaging capabilities to provide a medical device capable of navigating to peripheral airways and obtain biopsies in efficient and economical manners. In several embodiments, medical devices described hereby may be utilized to obtain biopsies of nodules in peripheral airways of lungs. FIG. 3B includes a front view of the distal end 316 of elongate members 302. More specifically, FIG. 3B illustrates a predefined rotational angle 324 between embedded imaging transducer 306 and pre-curved instrument 304 with respect to rotational imaging transducer 308. FIG. 3C includes radial image 318 generated by rotational imaging transducer 308 in conjunction with pre-curved instrument indicia 320, embedded transducer indicia 322, and predefined rotational angle 324. In the illustrated embodiment, the predefined rotational angle 324 is 180 degrees. However, in various embodiments, the predefined rotational angle 324 could be between 0 and 360 degrees. In several embodiments, the predefined rotational angle 324 may be a subset of between 0 and 360 degrees, such as between 90 and 270 degrees. In many embodiments, a predetermined rotation angle that results in the indicia obscuring the view of target tissue in the radial image may be avoided. For example, angles between 0 and 45 degrees and angles between 315 and 360 may be avoided. In some embodiments, the predefined rotational angle 324 may be between 120 and 240 degrees, such as 180 degrees.

In various embodiments, embedded imaging transducer 306 may extend through the field of view of rotational embedded imaging transducer 306 at the predefined rotational angle 324 with respect to the projected position of pre-curved instrument 304 extended out of elongate member 302. Additionally, embedded imaging transducer 306, or at least the portion extending through the field of view of rotational transducer 114 may be constructed from a material that will cause indicia in the radial image (e.g., a material that interacts with imaging energy emitted by rotational imaging transducer 308 in a characteristic way). In some embodiments, fiber optic cable of embedded imaging transducer 306 may provide the embedded transducer indicia 322. In one or more embodiments, the embedded transducer indicia 322 may comprise a shadow of the embedded imaging transducer 306 in radial image 318. In several embodiments, rotational imaging transducer 308 may be positioned within elongate member 302 to generate radial image 318 including embedded transducer indicia 322. Accordingly, an operator may determine a projected position of the pre-curved instrument 304 prior to extending the pre-curved instrument 304 out of the elongate member 302.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

All of the devices and/or methods disclosed and claimed hereby can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method disclosed hereby without departing from the concept, spirit and scope of the disclosure. All such similar substitutes and modifications apparent to those skilled in the art are deemed to be within the spirit, scope and concept of the disclosure as defined by the appended claims.

1. A medical device, comprising:
an elongate member having a proximal end, a distal end, a first lumen extending from the proximal end to a first distal opening proximate the distal end, a second lumen extending from the proximal end to the distal end, and an embedded transducer disposed in a wall of the elongate member, the embedded transducer positioned with a predefined rotational angle with respect to a projected position of an instrument extended out of a distal opening of the first lumen; and
a rotational transducer disposed in the second lumen, the rotational transducer configured to generate a radial image from within the second lumen, the radial image comprising indicia of the embedded transducer at the predefined rotational angle with respect to the projected position of the instrument.

2. The medical device of claim 1, the embedded transducer comprising at least a portion of an imaging transducer.

3. The medical device of claim 2, the embedded transducer comprising a distal portion of a fiber optic sensor.

4. The medical device of claim 1, the rotational transducer comprising a rotational imaging transducer.

5. The medical device of claim 4, the rotational imaging transducer comprising an ultrasound transducer.

6. The medical device of claim 1, wherein the embedded transducer is disposed within a wall of the second lumen of the elongate member.

7. The medical device of claim 1, wherein the second lumen is positioned in the elongate member between the first lumen and the embedded transducer.

8. The medical device of claim 1, wherein the instrument comprises a pre-curved instrument.

9. The medical device of claim 8, wherein the pre-curved instrument comprises one or both of a needle and an ablation probe.

10. The medical device of claim 8, wherein the elongate member is configured to bend in a first direction when a distal end of the pre-curved instrument is positioned a first distance from the distal opening of the first lumen.

11. The medical device of claim 10, wherein the elongate member is configured to straighten as the pre-curved instrument is moved from the first distance to the distal opening of the first lumen.

12. The medical device of claim 11, wherein the elongate member is configured to remain substantially straight when the pre-curved instrument is extended out of the distal opening of the first lumen.

13. The medical device of claim 1, wherein the predefined rotational angle ranges from about 45 to about 315 degrees.

14. The medical device of claim 1, wherein the elongate member has an outer diameter of less than 2 mm.

15. A system, comprising:
an elongate member having a proximal end, a distal end, a first lumen, a second lumen, and an embedded transducer;
an instrument disposed in the first lumen, wherein the embedded transducer is positioned with a predefined rotational angle with respect to a projected position of the instrument extended out of a distal opening of the first lumen; and
a rotational transducer disposed in the second lumen, the rotational transducer configured to generate a radial image from within the second lumen, the radial image comprising indicia of the embedded transducer at the predefined rotational angle with respect to the projected position of the instrument.

16. The system of claim 15, wherein the instrument comprises a pre-curved needle.

17. The system of claim 15, wherein the instrument comprises a pre-curved ablation probe.

18. A method, comprising:
inserting a distal end of an elongate member into a body lumen, the elongate member having a proximal end, the distal end, a first lumen, a second lumen, and an embedded transducer;
generating a radial image with a rotational transducer disposed in the second lumen of the elongate member, the radial image comprising indicia of the embedded transducer at a predefined rotational angle with respect to a projected position of an instrument extended out of a distal opening of the first lumen of the elongate member.

19. The method of claim 18, comprising rotating the elongate member to align a target tissue at the predefined rotational angle with respect the projected position of the instrument.

20. The method of claim 19, comprising extending the instrument out of the distal opening of the first lumen of the elongate member to obtain a biopsy of the target tissue.
